(19) United States
(12) Patent Application Publication
(10) Pub. No.: US 2016/0341741 A1
(43) Pub. Date: Nov. 24, 2016

(54) SCREENING FOR TARGET-SPECIFIC AFFINITY BINDERS

(71) Applicant: PETER C. KLOEHN, Herts (GB)

(72) Inventor: PETER C. KLOEHN, Herts (GB)

(21) Appl. No.: 15/112,575

(22) PCT Filed: Jan. 21, 2015
(86) PCT No.: PCT/EP2015/051146

§ 371 Date: Jul. 19, 2016

(30) Foreign Application Priority Data
Jan. 21, 2014 (GB) 1400962.5

(51) Int. Cl. G01N 33/68; C07K 16/18; C07K 16/10; C12N 15/113; C07K 16/00

(52) U.S. Cl. CPC G01N 33/6854; C12N 15/113; C07K 16/00; C07K 16/1063; C07K 16/18; C12N 2310/14; C07K 2317/30; C07K 2317/33

(57) ABSTRACT

A method for determining the specificity of one or more antibodies, or one or more alternative affinity binders, for a target antigen can include the following steps: (i) analyzing binding of the antibody, or the alternative affinity binder, to a target antigen expressed by an antigen-expressing cell; (ii) analyzing binding of the antibody, or the alternative affinity binder, to a target antigen expressed by an equivalent antigen-expressing cell, in which cell expression of the target antigen has been silenced and/or the gene of the target antigen has been deleted; and (iii) comparing the binding reactions of the antibody, or the alternative affinity binder, in steps (i) and (ii). In various embodiments, the method further features that a reduction or loss of binding in step (ii), compared to step (i), indicates that the antibody, or the alternative affinity binder, has affinity and/or specificity for the target antigen.

### CROSS REFERENCE TO RELATED APPLICATIONS

This application claims the benefit of Application Nos. PCT/EP2015/05146 filed on Jan. 21, 2015 and GB 1400962.5 filed on Jan. 21, 2014, the disclosures of which are all herein incorporated by reference in their entirety.

### FIELD OF THE INVENTION

In the present invention, a method of screening libraries of antibodies and alternative affinity binders for target-specific candidates is disclosed, which method uses gene silencing or deletion of the target-encoding gene in a high-throughput setting.

### DESCRIPTION

The invention relates to screening libraries of antibodies and alternative affinity binders. More particularly, the invention relates to the validation of antibody specificity and alternative affinity binder specificity by depleting the gene-encoded protein target an antibody or affinity binder has been raised against. In the following, the discussion related to antibodies will also apply to alternative affinity binders if not otherwise specified.

### BACKGROUND OF THE INVENTION

Antibodies are biological macromolecules, produced by the immune system of a host in response to foreign proteins (“targets”). Antibodies recognize a distinct part of the target, called an antigen. The establishment of technology to isolate and to develop antibodies in industry scale permitted the use of antibodies in academic research and in the clinic for the diagnosis and therapy of diseases.

Whilst natural antibodies recognize target proteins with high precision and efficacy, a process which is controlled by a complex regulatory network of the host's immune system, commercial antibodies produced with recombinant means often perform less reliable, a problem that has been raised in various reports [1-6].

### SUMMARY OF THE INVENTION

In conventional mAb development immune cells from spleens of immunized mice are fused with myeloma (B cell cancer) cells and subsequently antibody-secreting hybrid cells, so-called hybridoma cells, identified by enzyme-linked immunosorbent assay (ELISA). Where conventional mAb development aims to select high-affinity antibodies against the immunogen, the approach according to the present invention relies on the genetic deletion or depletion of the target antigen designed to identify target-specific antibodies. This is accomplished by testing each antibody against the plurality of proteins expressed in test cells which are deficient for the antibody target. Importantly, this technology can be combined with other high-throughput screening technologies, like yeast and ribosome display aimed to identify alternative affinity binders.

Further, false negative results during testing of antibody libraries may arise where an antigen is hidden (cryptic) under native conditions. We here disclose a method to modulate protein folding states globally in vitro, a method which allows validation of antibodies against cryptic antigens at high-throughput. This approach may also provide additional information on the binding properties of antibodies and may significantly reduce cost during antibody development. While binding of antibodies to the native state of proteins is for instance essential for their use in immunofluorescence, immunoprecipitation, immunohistochemistry, and of course therapy, the capability of binding to a denatured protein is a prerequisite for their use in Western Blotting. The modulation of protein folding states is achieved by incubation of antigen producing cells, or antigens as such, with denaturing agents, after which binding properties of antibodies will be tested subsequently. Where new antibodies are generally tested in a costly set of applications, the method according to the invention provides key information about antibody binding properties already at the screening stage, and is thus capable of predicting future application and reducing development costs.

### BRIEF DESCRIPTION OF THE DRAWINGS

FIG. 1: Antibody validation by RNA interference. To deplete Early Endosome Antigen 1 (EEA1), a protein marker of early endosomes, EEA1, HepG2 cells were transfected with siRNA against EEA1 and control (scrambled siRNA) and grown for three days. Cells were then fixed, permeabilized and co-labelled with antibodies against Tubulin and EEA1. Representative image for anti-Tubulin, anti-EEA1 and the merged images are shown. Cell nuclei, labelled with 4′,6-diamidino-2-phenylindole, dihydrochloride (DAPI) are shown in the merged image only. Scale bar: 5 μm.

FIG. 2: Distinct siRNAs against β-Catenin differ in their efficacy of gene silencing. HEK293 cells were transfected with three siRNAs against β-Catenin and cultured for 3 days. Cells were then fixed, permeabilized and labelled with a monoclonal anti-β Catenin antibody (clone 14/Beta-Catenin, BD Transduction Labs). Confocal images were taken for each condition and 20 frames analyzed using Volocity image analysis software and the normalized residual fluorescence signal (NRF) was determined. NRF values of 1, 0.05, 0.15, 0.25 were calculated for siRNA control, siRNA1 b-catenin, siRNA2 b-catenin and siRNA3 b-catenin, respectively. Scale bar: 5 μm.

FIG. 3A-D: Validation of antibody specificity by immunofluorescence (IF) and Western Blotting. HepG2 cells (FIG. 3A, FIG. 3B) and SHSY5Y (FIG. 3C, FIG. 3D) cells were transfected with siRNA against Lamp1 (FIG. 3A, FIG. 3B) and Thy1 (FIG. 3C, FIG. 3D), and grown on glass cover slips and 10 cm petri dishes. After 3 days cells grown on glass cover slips were fixed and permeabilized. Cells grown in 10 cm dishes were lysed in RIPA buffer for 20 min on ice and further processed as described in Methods. Scale bar: 5 μm.

FIG. 4: Validation of the specificity of an anti-Jun antibody. HeLa cells, transfected with siRNA against c-Jun (siRNA1 c-Jun) and scrambled control siRNA (siRNA control) were grown for three days. Cells were then fixed, permeabilized and labelled with an anti-Jun antibody (clone 3/Jun, BD Transduction Labs) and DAPI (Nuclei). The complete loss of antibody signal in cells treated with siRNA c-Jun confirms the specificity of the anti-Jun antibody. Scale bar: 5 μm.

FIG. 5A-C: Validating the specificity of ERK2 antibodies using CRISPR Cas. To validate commercial anti-ERK2 antibodies the protein kinase gene ERK2 was deleted in HAP1 cells using CRISPR/Cas9. (FIG. 5A) To ensure that ERK2, but not ERK1 is deleted in HAP1 cells, lysates of wild-type and ERK2 knockout cells were separated using Western Blotting. After incubation with an antibody against ERK1/2 (anti-ERK1/2, CST 9102) a double band is detected in wild-type cells, whereas in ERK2 knockdown cells the lower ERK2 band is not detected, confirming ERK2 depletion. (FIG. 5B) To test the quality of commercially available anti-ERK2 antibodies blots were incubated with a 1:1000 dilution of the anti-ERK2 antibodies specified. Three out of four antibodies show off-target effects. (FIG. 5C) Wild-type and ERK2 knockout cells were incubated with the target-specific antibody anti-ERK2 (Bethyl, A302061A) followed by DyLight 488-conjugated anti-rabbit antibody (H+L) and DAPI (Nuclei). The specificity of the antibody is confirmed by a complete loss of the ERK2 label in ERK2 knockout cells. Scale bar: 5 μm.

FIG. 6A-B: Validating the specificity of an antibody against a cryptic epitope of Death-associated protein 3 (DAP3) by in situ denaturation. (FIG. 6A) HeLa cells, transfected with siRNA against DAP3 and with scrambled RNA control (siRNA control) were grown for three days. Cells were lysed and proteins separated by electrophoresis. After blotting onto PVDF membranes, blots were labelled with an antibody raised against the peptide NPSLLERHCAYL of DAP3 (EB05427, Everest Biotech). (FIG. 6B) In situ denaturation of fixed cells with guanidinium thiocyanate (GTC) reveals a mitochondrial label of DAP3 and thus permits to test the specificity of the anti-DAP3 antibody by immunocytochemisty. Scale bar: 5 μm.

FIG. 7: Detection of a cryptic actin epitope by in situ denaturation of cells. SHSY5Y cells were plated onto glass cover slips and grown for 3 days in a humidified CO2 incubator at 37° C. After fixation, cells were permeabilized and treated with 4M urea for 10 minutes. After thorough washing, cells were labelled with a mouse anti-Actin antibody (clone 2Q1055, Abcam) for 1 hour at room temperature. After washing twice with PBS, cells were labelled with DyLight 488-conjugated anti-mouse antibody (H+L) and DAPI (Nuclei) for 1 hour. Representative confocal images under native and denaturing conditions are shown. Scale bar: 5 μm.

FIG. 8A-B: Detection of cryptic Protein Disulphide Isomerase (PDI) and Histone H3 binding sites (cryptic epitopes) by in situ denaturation. SHSY5Y cells were plated onto glass cover slips and grown for 3 days in a humidified CO2 incubator at 37° C. After fixation, cells were permeabilized and treated with 3.5M GTC for 10 minutes. After thorough washing with PBS, cells were labelled with anti-PDI and anti-Histone H3 antibody for 1 hour at room temperature (FIG. 8A). After 2 washes with PBS, cells were labelled with DyLight 488-conjugated anti-mouse antibody (H+L) and DAPI (Nuclei) for 1 hour (FIG. 8B). Representative confocal images under native and denaturing conditions are shown. Scale bar: 5 μm.

FIG. 9: Detection of a conformational Nucleoporin 98 kDa binding site (conformational epitopes) by in situ denaturation. SHSY5Y cells were plated onto glass cover slips and grown for 3 days in a humidified CO2 incubator at 37° C. After fixation, cells were permeabilized and treated with 4M urea for 10 minutes. After six washes with PBS, cells were labelled with anti-Nucleoporin 98 kDa antibody (clone C39A3, CST) for 1 hour at room temperature. After 2 washes with PBS, cells were labelled with DyLight 488-conjugated anti-rabbit antibody (H+L) and DAPI (Nuclei) for 1 hour. Representative confocal images under native and denaturing conditions are shown. A more than 90% decrease in fluorescence intensity confirms that the targeted Nucleoporin 98 kDa epitope is highly sensitive to protein conformational changes. Scale bar: 5 μm.

FIG. 10A-B: Tubulin and EEA1 binding sites detected under native and denaturing conditions are invariant to protein conformational changes. SHSY5Y cells were plated onto glass cover slips and grown for 3 days in a humidified CO2 incubator at 37° C. After fixation, cells were permeabilized and treated with 4M urea for 10 minutes. After six washes with PBS, cells were labelled with (FIG. 10A) anti-Tubulin antibody (clone DM1B, Abcam) and (FIG. 10B) anti-EEA1 (clone C45B10, CST) antibody for 1 hour at room temperature. After 2 washes with PBS, cells were labelled with DyLight 488-conjugated antibody (H+L) and DAPI (Nuclei) for 1 hour. The targeted binding sites are detected under native and denaturing conditions. To clearly show the presence of tubulin fibres, mitotic cells were included in A. Scale bar: 5 μm.

FIG. 11A-D: Schematic representation of the Epitope Conformation Switch (ECS) assay and its use in the high-throughput characterization of antibody libraries. (FIG. 11A, FIG. 11B) In a typical screening protocol, cells are grown to confluence in 96-well plates, followed by fixation and permeabilisation. Plates are treated with (FIG. 11A) PBS or (FIG. 11B) denaturing agents, followed by thorough washing with PBS. After incubation with aliquots of antibodies from antibody libraries or hybridoma supernatants for 1 h, cells are washes 3-4 times with PBS and labelled with fluorescent-conjugated secondary antibodies. (FIG. 11A, FIG. 11B) Grey dots represent positive wells of fluorescently labelled cells, whereas white wells are negative. Note that some wells are positive (negative) under native (FIG. 11A) and denaturing (FIG. 11B) conditions, respectively, in analogy to antibodies represented in FIGS. 7-11. (FIG. 11C) The identification of antibodies against cryptic or conformational epitopes is shown in the differential readout. Antibodies that are positive in presence and absence of denaturing agents are denoted ‘invariant’. (FIG. 11D) Integration of the ECS assay into high-throughput screening of antibody libraries. As in situ denaturation is critical to identify antibodies against cryptic and conformational epitopes the ECS assay is the primary assay for the characterization of binders, followed by testing of antibody specificity and affinity, respectively.

FIG. 12: Isolation of target-specific monoclonal antibodies. The isolation of monoclonal antibodies can be divided into 5 stages. The typical duration of each stage is specified. Immunization of the host with recombinant protein or peptide (A) is followed by the fusion of isolated B lymphocytes with myeloma (B cell cancer) cells and transferred into wells of a 96-well plate (B). The fused cells, termed hybridoma cells, are grown for typically two weeks in a heterogeneous mix of cells before antibody-producing cells are identified (C). During this phase hybridoma supernatants are retrieved and analyzed in regards to their antibody isotypes and target specificity. Subsequently, selected antibody-producing hybridoma cells are subcloned to obtain monoclonal hybridoma cells (D) and monoclonal antibodies are isolated and purified (E).

### DETAILED DESCRIPTION OF THE INVENTION

The invention provided a method for determining the specificity of one or more antibodies, or one or more alternative affinity binders, for a target antigen, comprising of the following steps:
(i) analyzing binding of the antibody, or the alternative affinity binder, to a target antigen expressed by an antigen-expressing cell;
(ii) analyzing binding of the antibody, or the alternative affinity binder, to a target antigen expressed by an equivalent antigen-expressing cell, in which the target antigen has been transcriptionally silenced, and/or deleted; and
(iii) comparing the binding reactions of the antibody, or the alternative affinity binder, in steps (i) and (ii)
   wherein a reduction or loss of binding in step (ii), compared to step (i), indicates that the antibody, or the alternative affinity binder, has affinity and/or specificity for the target antigen.

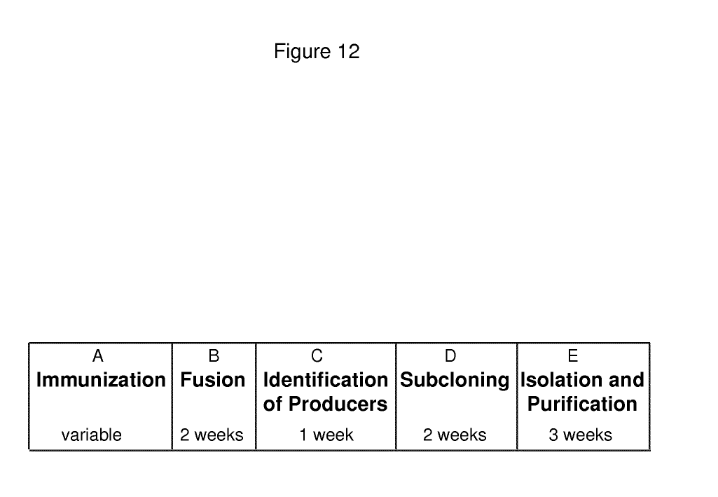

The use of transcriptional silencing or deletion of the target-encoding genes for the identification of target-specific antibodies has particular surprising advantages. According to the state of the art, for the generation of a target specific antibody animals are immunized with a peptide or recombinant protein. After several weeks splenocytes are isolated, fused with mouse or rabbit myeloma cells and transferred into wells of a cell culture plate. In FIG. 12 a general time-line of monoclonal antibody isolation according to the state of the art is depicted.

Usually 10-20 multi well plates (comprising between about <500 and >2000 well each) are plated with hybridoma cells. Importantly, each well contains a variety of cells, including myeloma cells, hybridoma cells and splenocytes and the subsequent steps are critical to identify an antibody-producing hybridoma clone. After expansion of cells by cell division for typically two weeks there is a time window of 1 week to select a limited number of wells to take forward for subcloning (Identification of producers). The most commonly used assay to identify antibody-producing hybridomas is an ELISA with the peptide/recombinant protein that has been used for immunization. A second assay may identify the antibody isotype of producers.

The decision for selecting hybridomas is therefore based on: (a) Quantity of antibodies secreted and their putative affinities, and (b) antibody isotypes. Importantly, the specificity of antibodies is uncharacterised at this stage. Even though a secreted antibody may bind to the immunogen, its binding affinity to a multitude of other related or unrelated proteins which constitute the proteome remain untested.

In one preferred embodiment of the present invention the screening of a library of monoclonal antibodies or alternative affinity binders, against a pair of cells is provided, one of which expresses the antibody target, while in the other one the expression of said target has been partially or completely depleted. This approach is preferably carried out in a high content screening or high throughput screening approach.

The value of transcriptional silencing or deletion of the target-encoding gene for the validation of antibodies has recently been suggested.

Mannsperger et al. disclose an RNA interference (RNAi)-based validation of antibodies using reverse phase protein arrays (RPPA) [7]. This approach follows the idea of a dot-immunoblot, in which large numbers of samples taken from cellular lysates are arrayed on solid phase carriers. The aim of this approach is to display as many proteins of a cellular proteome as possible (as it is the object in RPPA). The approach however is unsuitable for the screening of an antibody library against a particular target expressed in a given cell.

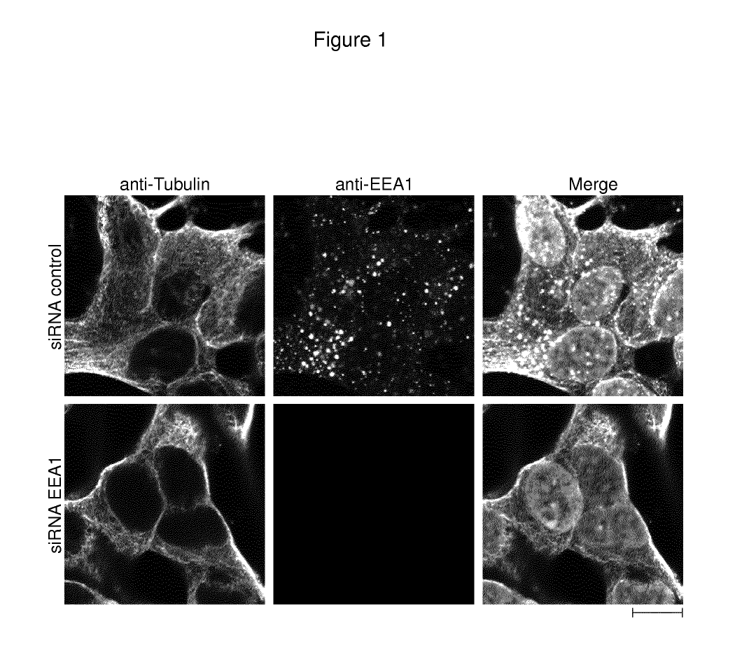

RPPA require serial dilutions of control cell lysates which are spotted along with dilution series prepared from siRNA-treated samples and greatly limit the throughput where antibody libraries comprise hundreds or thousands of antibodies. Furthermore, chemiluminescence-based assays are hampered by their low dynamic range and require time lapse exposure to avoid signal saturation. Laser-scanning microscopy based procedures retain the integrity of cells, a compelling advantage over analysis of cell lysates, since compartmentalization and structural features of proteins contribute additional criteria to assess the specificity of antibodies (FIGS. 1-6). Lastly, where experimental variation and pipetting errors affect the statistical analysis of RPPA, the analysis of thousands of cells during high content analysis is providing statistical power whilst pipetting errors are excluded.

Stadler et al. disclose a validation approach of antibody binding and protein subcellular localization using RNAi and confocal microscopy [8]. In this paper, 75 polyclonal antibodies targeting proteins encoded by 65 different genes were characterized with respect to their target-binding properties. The authors claim that such an approach is of great importance under the continued work of mapping the human proteome on a subcellular level. Stadler et al. do not use RNAi to screen antibody and affinity binder libraries, respectively, but instead validate a catalogue of 75 antibodies. Hence, Stadler et al. characterize each polyclonal antibody against its target. However, the generic use of RNAi for antibody validation has a number of limitations that can be circumvented by deletion of the target-antigen encoding gene using CRISPR/Cas or TALEN. Firstly, highly transcribed genes produce high levels of mRNA which may result in insufficient levels of knockdown by RNAi, as the destruction of already transcribed mRNAs prior to their translation becomes rate-limited. This may in fact explain the inherent incompleteness of protein depletion by RNAi [9]. In these cases, direct targeting of the gene by CRISPR/Cas or TALEN is a more promising approach. Secondly, RNAi is limited to particular organisms that have a functional gene silencing machinery [10] which has been reported to be frequently compromised in some cancers [11-14] and in immortal cell lines, isolated thereof, respectively. The combined use of transcriptional silencing by RNAi and deletion of the gene-encoded protein by CRISPR/Cas or TALEN is therefore best suited to mitigate the shortcomings of RNAi.

Jensen et al. tested the specificity of ten commercial antibodies against al-adrenergic receptor (AR) using protein separation by Western blotting of heart and brain tissue from wild-type (wt) and AR knockout (ko) mice and found that none of the antibodies were specific for AR [3]. Notably, this approach is limited to gene-depletion in animals and antibodies against human antigens may not be tested due to sequence variation. The resent establishment of genome editing by CRISPR/Cas and Talen, however, provides a convenient approach for the deletion of specific genes in human cell lines.

Generally, it must be stated in this context that the identification of an antibody or affinity binder from a cloud of unknown binders is not trivial, and requires way more than what can be learned from validation processes related to existing antibodies.

The translation of methods to silence or delete the target-encoding gene to monoclonal antibody development, as set forth in the present invention, harnesses high-throughput screening to validate commercial polyclonal antibodies, for the development of target-specific monoclonal antibodies. Monoclonal antibodies are “renewable biologics”, and as such, offer several advantages over polyclonal antibodies, like their low batch-to-batch variation, but their development is greatly hampered by unexpected cross-reactivity [15-19].

Thus, the use of loss-of-target approaches as proposed here greatly improves quality control at early stages of mAb development. Whilst the affinity of monoclonal antibodies is typically well characterized, their specificity (i.e., their propensity to bind to the target protein, but not to other proteins represented in the proteome) remains untested. Although mAbs are generally considered highly specific, the occurrence of polyspecificity and cross-reactivity are observed frequently [15,16]. In addition, antibodies with high affinity do not necessarily guarantee specificity. In fact, several research groups have pointed out that antibodies of low affinity tend to discriminate better between two antigens than antibodies of high affinity [17]. As a result, lower-affinity antibodies are more discriminatory, which makes them more specific than high-affinity antibodies [17]. By including a loss-of-target approach on the context of the whole proteome of test cells, the specificity, cross-reactivity and affinity of antibodies can be conveniently tested within a very short time.

The screening approach described here goes beyond the mere validation of antibody specificity. It is designed to select superior monoclonal antibodies by screening libraries of antibodies and alternative affinity binders against a sole target using RNAi and genome editing technology, and introduces four main advantages as compared to prior art.

Firstly, this platform permits the identification of binders with distinct affinities and specificities, selected from a large pool of antibodies or alternative affinity binders. Secondly, it allows screening of antibody libraries against epitopes that are cryptic in cells under native conditions. Thirdly, additional information on antibody binding properties, analyzed by in situ modulation of protein folding states during antibody screening allows prediction of the binding characteristics of affinity reagents in a targeted approach. Fourthly, where use of RNAi is insufficient the genetic depletion of the antigen target can be performed by genome editing technology.

For instance, affinity reagents which only bind to the denatured form of the antigen may not be suitable for applications like immunoprecipitation and immunohistochemistry and may be missed during high content screening of cells under standard conditions. The disclosed approach allows screening of affinity reagents with defined characteristics and greatly reduces the cost of subsequent antibody validation. In summary, this approach provides a powerful tool to develop monoclonal antibodies from a given library against a specific target. This approach may thus be used to detect antibody candidates suitable for either therapy or diagnostics.

According to another preferred embodiment of the present invention, the transcriptional silencing of the target antigen, or the deletion of the target antigen, is accomplished by the application of at least one method selected from the group consisting of
RNA-mediated interference (RNAi) (transcriptional silencing of the target antigen)
CRISPR/Cas technology (deletion of the target antigen-encoding gene)
Talen technology (deletion of the target antigen-encoding gene)
Zn finger nuclease technology (deletion of the target antigen-encoding gene)

RNAi, or gene silencing, is a conserved biological process which controls gene expression in cells of living organisms by destroying specific RNA templates, the messenger RNA (mRNA), by short double-stranded RNA molecules, termed short interfering RNA (siRNA) [20]. This biological principle has been harnessed to silence gene expression in mammalian cells by synthetic siRNA molecules [21,22].

Before the pivotal role of siRNA was discovered, unresolved mechanisms of gene expression control had been termed post transcriptional gene silencing (PTGS), co-suppression, and quelling. Only after the molecular mechanism of RNAi were fully understood did it become clear that these apparently unrelated processes all described the RNAi phenomenon. Andrew Fire and Craig C. Mello shared the 2006 Nobel Prize in Physiology or Medicine for their work on RNAi in the nematode worm Caenorhabditis elegans, published in 1998 [20].

The RNAi pathway is found in many eukaryotes and is initiated by the enzyme Dicer, which cuts long double-stranded RNA molecules into shorter fragments of 21-23 base duplexes. Each siRNA molecule is split into two single-stranded RNAs, the passenger and the guide strands. While the passenger strand is degraded, the guide strand is incorporated into the RNA-induced silencing complex (RISC), resulting in post-transcriptional gene silencing. This occurs when the guide strand pairs with a complementary sequence in a messenger RNA molecule and is sliced by Argonaute, a catalytic component of the RISC complex.

The RNAi pathway is often harnessed in experimental biology to study the function of genes in vitro and model organisms in vivo. Double-stranded RNA has sequence complementarity with a gene of interest and when introduced into a cell or organism, it is recognized as exogenous genetic material and activates the RNAi pathway. This mechanism can therefore be exploited to cause a drastic decrease in the expression of a targeted gene and may tell us something about the physiological role of the gene product. Since RNAi may not completely abolish expression of the gene, this process is sometimes referred to as “knockdown”, to distinguish it from “knockout” processes in which the corresponding gene is entirely eliminated.

RNAi is particularly useful for genomic mapping and annotation and it may be possible to exploit RNAi for therapy. Among the first applications of RNAi in clinical trials were in the treatment of macular degeneration and respiratory syncytial virus.

RNAi in vivo delivery to tissues still poses a major challenge. To date, RNAi delivery is only applicable to surface tissues such as the eye and respiratory tract. However, this problem does not occur in the screening approach that is subject of the present invention, where the inventors demonstrate that the method is fully workable.

The inventors have thus found, surprisingly, that RNAi offers a useful tool to transcriptionally silence a gene that encodes for a given target of interest for which the present invention seeks to find antibodies or alternative affinity binders encompassed in a given library.

Whilst RNAi affects gene expression by interfering with the transcribed mRNA of a gene, the coding sequence of a gene itself can be targeted with emerging technologies. RNA-guided nucleases have revolutionized the way researchers can modify, repair or delete genetic information. Present in many bacteria and most archaea, CRISPRs (clustered regularly interspaced short palindromic repeats) are specific DNA loci which contain short repetitions of base sequences. Each repetition is followed by short segments of “spacer DNA” and is thought to be integrated into the DNA from previous exposures to a virus.

Of the currently evolving genome editing technologies, the class of RNA-guided endonucleases known as Cas9 is the most rapidly developing system. The CRISPR/Cas system which is thought to act as a prokaryotic immune system by conferring resistance to foreign genetic elements is a programmable nuclease, guided by the crRNA and tracrRNA (or trans-activating crRNA) to cleave specific DNA sequences in a manner analogous to RNAi in eukaryotic organisms.

The inventors have thus found, surprisingly, that CRISPR/Cas offers another useful tool to delete a gene that encodes for a given target of interest for which the present invention seeks to find antibodies or alternative affinity binders encompassed in a given library.

Zinc-finger nucleases (ZFNs), a class of engineered DNA-binding proteins are generated by fusing a zinc finger DNA-binding domain to a DNA-cleavage domain and facilitate targeted editing of the genome by creating DNA double-strand breaks at unique sequences within complex genomes. By harnessing the endogenous DNA repair machinery, ZFNs can be used to precisely modify the genomes of higher organisms.

A general way to introduce a site-specific double-strand break (DSB) is catalysed by ZFNs that combine the non-specific cleavage domain of FokI endonuclease with zinc finger proteins (ZFPs). Subsequently the DSB is being repaired by either homologous recombination or non-homologous end joining.

ZFNs have become useful reagents for manipulating the genomes of many model organisms, including Drosophila melanogaster, Caenorhabditis elegans, zebrafish, mice, rats, rabbits, and various types of mammalian cells. ZFNs have recently been used to permanently remove essential splicing sequences in exon 51 of the dystrophin gene, a therapeutic intervention that restored the dystrophin reading frame in ˜13% of Duchenne muscular dystrophy (DMD) patient mutations (Mol Ther., 2014, doi: 10.1038/mt.2014.234). An ongoing clinical trial evaluates the feasibility of ZNFs that disrupt the CCR5 gene in CD4+ human T-cells as a potential treatment for HIV/AIDS.

The inventors have thus found, surprisingly, that ZNFs offer another useful tool to delete a gene that encodes for a given target of interest for which the present invention seeks to find antibodies or alternative affinity binders encompassed in a given library.

Transcription activator-like (TAL) effectors are newly described DNA binding proteins that have been utilized to generate site-specific gene editing tools by fusing target sequence-specific TAL effectors to nucleases (TALENs).

Produced by plant pathogenic bacteria, TAL effectors directly modulate host gene expression. Upon delivery into host cells via a bacterial secretion system, they translocate into the nucleus, bind to effector-specific sequences in host gene promoters and activate transcription. TAL effector binding to DNA is mediated by a highly conserved region of these proteins that contains up to 28 tandem repeats of a 33- to 35-amino-acid-sequence motif. Sequence variation among the repeats is almost exclusively localized to a pair of residues at positions 12 and 13, called the repeat-variable di-residue (RVD). Using the RVD code, targets of new TAL effectors have been correctly predicted and have thus allowed for the engineering of specific DNA binding domains.

Targeted DNA DSB mediated by TALEN can be utilized for (a) introduction of small genomic mutations; (b) introduction of endogenous tags into proteins; (c) introducing mutations resulting in frame-shifts or stop-codons and (d) excision and repair of mutations by homologous recombination after DSB. TALENs have been used to generate stably modified human embryonic stem cell (hESC) and induced pluripotent stem (iPS) cells, as well as knock-in cells and animals, respectively.

A variety of examples of TALENs and CRISPR/Cas9-mediated genome editing in human cells have been published, including gene addition in hESCs (AKT2) and K562 cells (CCR5), gene corrections in iPS cells (OCT4, PITX3, AAVS1) and gene disruptions in iPS cells (CIITA) and hESCs (SORT1, ATGL, GLUT4). Where genetic mutations are associated with disease, such as Sickle cell anemia, Xeroderma pigmentosum, and Epidermolysis bullosa TALENs and CRISPR/Cas may be used to correct the genetic defects. However, applications in gene therapy for TALENs and CRIPSR/Cas are currently limited by a number of obstacles, including the lack of an efficient delivery mechanism, uncertainty in the specificity of DNA binding, and unknown immunogenic factors. However, these problems are irrelevant in the screening approach that is subject of the present invention, which is in vitro and does not require targeted in vivo delivery systems.

The inventors have thus found, surprisingly, that TALENs offers a useful tool to delete a gene that encodes for a given target of interest for which the present invention seeks to find antibodies or alternative affinity binders encompassed in a given library.

According to a preferred embodiment of the invention, the binding of the antibody or the alternative affinity binder in steps (i) and/or (ii) is analyzed directly on or in the antigen-expressing cell.

According to another preferred embodiment of the invention, the binding in steps (i) and/or (ii) is studied in a lysate prepared from the antigen-expressing cell.

According to still another preferred embodiment of the invention, the antigen-expressing cell is generated by introducing a gene encoding the target antigen under the control of a constitutive or an inducible promoter.

According to another preferred embodiment of the invention at least one antigen-expressing cell, or the antigens expressed by said cell, is/are treated with a denaturing agent before the binding of the antibody or the alternative affinity binder in steps (i) and/or (ii) is analyzed.

This approach is also called Epitope Conformation Switch™ assay.

The interaction between paratope (i.e., the part of an antibody which recognizes an antigen) and epitope (i.e., the part of the antigen that is actually recognized by the antibody) not only depends on the equilibrium affinity of antibody-epitope bonds, but also on the protein folding state of the epitope. The latter determines the choice of applications an antibody can be used for. For instance, antibodies which bind exclusively to the native conformation of a protein target can be used for therapy, immunoprecipitation or immunohistochemistry, but not for routine Western Blotting where proteins are separated typically in their denatured form. The In situ denaturation of cells, or the antigens expressed therein, in presence and absence of denaturing agents as described in this invention instead allows to test whether an antibody binds to antigens in its native or denatured, or to both states. This assay therefore permits the targeted selection of monoclonal antibodies in respect to their future application, is predictive and cost-saving. It furthermore allows screening of antibody libraries against cryptic epitopes.

Preferably, said method serves to find antibodies or affinity binders that meet at least one criterion from the following list:

a) they are specific and bind to cryptic epitopes of a target inaccessible in its native conformation.
b) they are specific and bind conformational epitopes, and/or
c) they are specific and are invariant to conformational changes of the epitope.

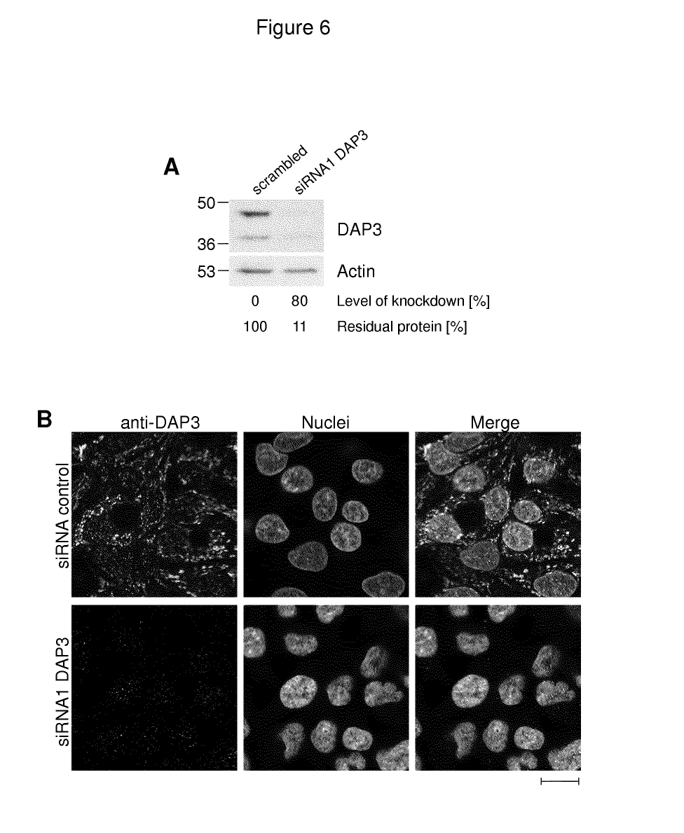

Cryptic epitopes are epitopes that are inaccessible from outside a regularly folded protein, either by conformational constraints or by shielding due to post-translational modifications, but that become exposed after denaturation. This means that antibodies or affinity binders against cryptic epitopes can only bind these epitopes under denaturing conditions, but not under native conditions. According to this specific embodiment, antibody libraries are screened in presence and absence of denaturing agents to identify cryptic epitopes which can only be bound under denaturing conditions, but not under native conditions [23-26]. Antibodies and affinity binders against cryptic epitopes are otherwise overlooked during screening of the respective libraries, since they are not detected under native conditions. Such antibodies or affinity binders can be used in association with conformation-independent antibodies or affinity binders to test in vitro and in vivo the presence of protein and accessibility to cryptic epitope, respectively (FIG. 6-8) [27-29].

Cryptic epitopes play a critical role during development of autoimmune diseases [30-33]. Therefore, antibodies or alternative affinity binders against cryptic epitopes can be applied in therapy as well as in diagnostics, or other non-clinical applications.

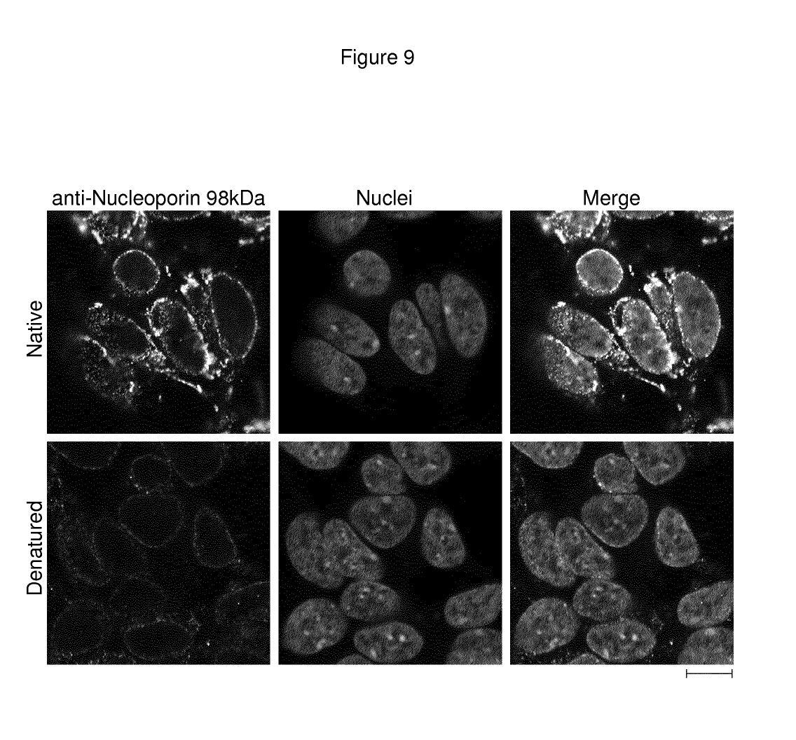

A conformational epitope (also called discontinuing epitope) is a sequence of sub-units from different stretches of a protein, which subunits compose an antigen under conditions when the protein is properly folded. Usually, an antibody or affinity binder against such conformational epitope can only bind the epitope under native conditions, while, under denaturing conditions, the epitope becomes disrupted and thus invisible for the antibody (FIG. 9). Thus, antibodies or affinity binders which only bind a conformational epitopes are not suitable for Western Blotting, in which the protein to be analyzed are denatured. Thus, being able, already at the stage of a screening process, to determine whether an antibody binds only the conformational epitope, is extremely helpful.

Antibodies against invariant epitopes can bind a target epitope under native and denaturing conditions. These antibodies or affinity binders can be universally used in diagnostic and scientific applications like Immunoprecipitation (IP), Immunocytopchemistry (ICC), Immunohistochemitry (IHC) and/or Western Blotting (WB). They can likewise be used in therapeutic applications.

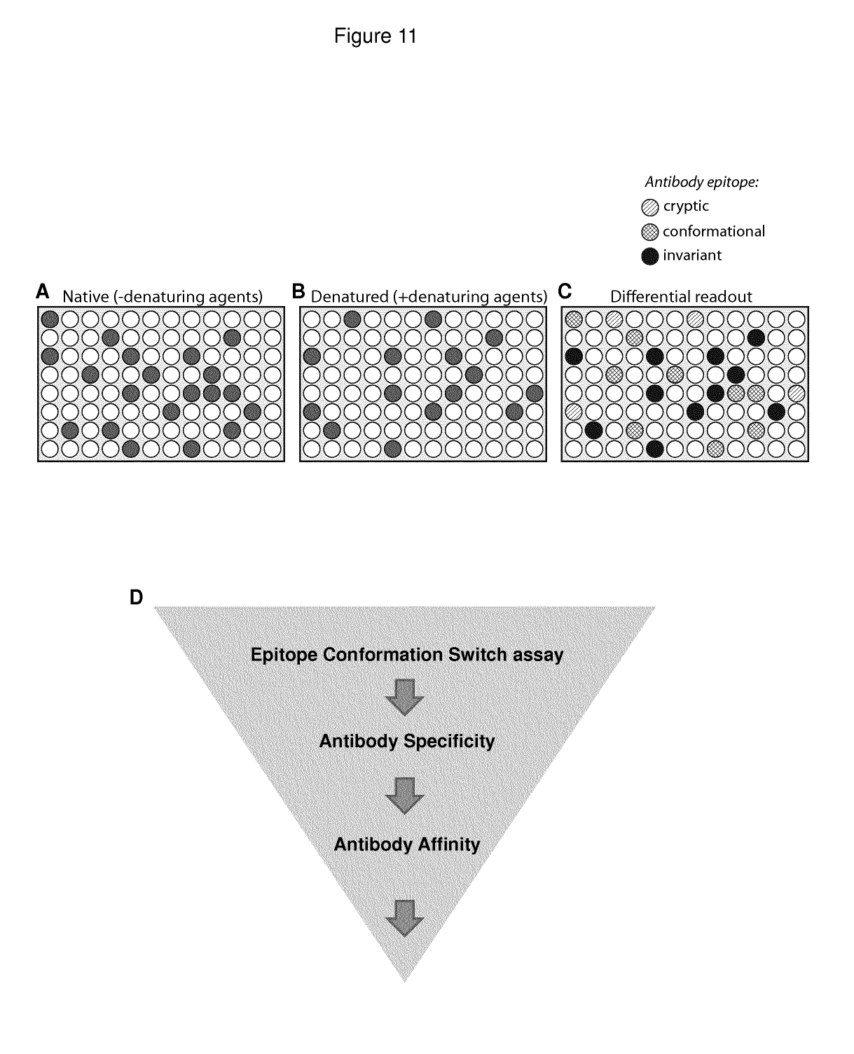

The characterization of antibody binding, or binding of alternative affinity binders, to epitopes under denaturing and native conditions has thus far not been disclosed in the context of antibody characterisation, or characterization of affinity binders. In fact, high-throughput screening of libraries according to 3 quality control criteria ((i) specificity and (ii) affinity of binders, and (iii) the nature of the epitope), is generally the first guiding approach for the identification of superior binders prior to selection/subcloning (FIG. 11).

While there is prior art in identifying cryptic epitopes [34], these studies do not disclose use of a conformational switch to identify antibodies or alternative binder against cryptic epitopes from antibody libraries.

Other assays to characterize proteins in live and fixed cells have been described. The In-Cell Western™ Assay from Li-Cor is mainly used to characterize protein activation, but does not use denaturation as in this invention to distinguish conformational from linear epitopes [35].

In summary, this approach leads to an unprecedented characterisation of antibodies by screening a cloud of unknown binders. It shows several advantages to prior art:

(a) High-content screening of antibodies is critical to identify superior antibodies from a cloud of unknown binders and outperforms antibody validation by Western blotting [6], Reverse Phase Protein Arrays [7] by throughput, dynamic range and sensitivity.
(b) The screening of antibody libraries using the Epitope Conformation Switch™ assay combined with genetic depletion of the target antigen identifies target-specific antibodies against cryptic, conformational and invariant epitopes and has not been described before.

According to still another preferred embodiment of the invention, one or more antibodies, or one or more alternative affinity binders, are provided by a library of cells. Preferably, the library of cells is at least one selected from
a library of hybridoma cells
a naive antibody library
a synthetic (combinatorial) antibody library, and/or
a synthetic (combinatorial) library of alternative affinity binders.

Thus, the library used may consist of a large variety of monoclonal antibodies or alternative affinity binders, produced either artificially, or taken from a naive immunoglobulin genome.

Because the library consists of so many variants, a sufficient degree of likelihood exists that it encompasses antibodies which bind to all conceivable biological targets.

This approach provides a powerful tool to identify and characterize monoclonal antibodies from a given library for their binding to a specific target. This approach may thus be used to detect antibody candidates suitable for either therapy or diagnostics.

Furthermore, a method for screening a plurality of cells for their capacity to secrete an antibody or an alternative affinity binder is provided. Said antibody or alternative affinity binder has affinity and/or specificity for a target antigen. The method comprises comparing the affinity and/or specificity of each antibody or alternative affinity binder produced from the plurality of cells using a method according to the above description.

Likewise, a method for selecting a cell line which produces an antibody or an alternative affinity binder is provided. Said antibody or alternative affinity binder has affinity and/or specificity for a target antigen which method comprises screening a plurality of cells by a method according to the above description, and selecting an antibody or an alternative affinity binder which shows a reduced or absent binding in step (ii) compared to step (i).

Preferably, in that method the cell line is a hybridoma. More preferably a plurality of hybridoma cells is produced by immunizing an animal with the target antigen, isolating a plurality of antibody-producing cells from the immunised animal and fusing the plurality of antibody-producing cells from the immunised animal and fusing the plurality of antibody-producing cells with an immortal cell type.

In a particularly preferred embodiment, the binding of the antibody or alternative affinity binder is analyzed by at least one method selected from the group consisting of
Immunofluorescence,
Western blotting,
Enzyme-linked immunosorbent assay (ELISA), and/or
surface plasmon resonance (SPR) based technology.

Most preferably, the antibody or alternative affinity binder found is used for at least one task selected from the group consisting of
Therapeutic purposes
Scientific purposes, and/or
Diagnostic or forensic purposes

Furthermore, a therapeutic, scientific, diagnostic and/or forensic antibody or alternative affinity binder found with a method according to the above description is provided.

In this respect, experiments with cynomolgus monkeys have shown higher than expected clearance rates of antibodies in therapeutic development [36]. The authors of the study conclude that off-target binding was presumed to account for the fast clearance and suggest that their in vivo assay is a test for risk mitigation. The method according to the present invention provides the potential to exclude off-target effects without the requirement for animal experiments. Therefore, the method allows the development of antibodies or affinity binders with better clinical performance.

In science, diagnostic and forensic these antibodies are preferably used for Immunoprecipitation (IP), Immunocytopchemistry (ICC), Immunohistochemitry (IHC) and/or Western Blotting (WB).

### DEFINITIONS

The term “antibody”, as used herein, relates to target binding proteins derived from the universal Immunoglobulin template. This term encompasses polyclonal and monoclonal antibodies as well as murine, chimerized, humanized and human antibodies. The term also encompasses different formats, like IgG, Fab, Fab2, scFv, domain antibodies, VHH and the like.

The term “affinity binders” encompasses all reagents with affinities to a target as specified below. The term “alternative affinity binders” (also called “antibody mimetics” or “alternative scaffolds”) relates to target binding proteins that have not been derived from the universal immunoglobulin template. Examples are Ankyrin Repeat Proteins (“DARPins”), C-Type Lectins (“Tetranectins”), A-domain proteins of Staphylococcus aureus (“Affibodies”), Transferrins (“Transbodies”), Lipocalins (“Anticalin”), Fibronectin (“AdNectins”), Kunitz domain protease inhibitors, Gamma crystallins or ubiquitins (“Affilin”), Cysteine knots (“Knottins or “Microbodies”), thioredoxin A scaffolds (“peptide aptamers”) or Target specific proteases obtained by directed evolution (“Alterases”).

The term “denaturing agent”, as used herein, relates to agents that are capable of denaturing proteins. Examples thereof encompass include guanidine salts, particularly guanidine hydrochloride, urea, and detergents such as sodium dodecyl sulfate (SDS) or Triton X 100.

The term “target antigen”, as used herein, relates to the “antigen” (any structural substance which serves as a target for the receptors of an adaptive immune response) an antibody is targeted against.

### EXAMPLES

Additional details, features, characteristics and advantages of the object of the invention are disclosed in the description of the respective figures and examples, which, in an exemplary fashion, show preferred embodiments of the present invention. However, these drawings should by no means be understood as to limit the scope of the invention.

The following protocol is suitable for gene silencing of cells in one 96-well plate with a total volume of 300 μl per well. The choice and volume of a transfection agent is cell-dependent and is optimised for each cell line. Ranges of typical volumes are given. Cells are plated into a 96-well plate at a concentration of 10⁵ cells/ml of medium and placed into a humidified CO2 incubator overnight to allow cells to adhere. After 12-16 h transfection complexes are formed by adding 5-40 μl siRNA or scrambled control RNA (20 nM) and 10-60 μl transfection agent to 300 μl fetal bovine serum (FBS)-free medium and mixed. After 20 min incubation at room temperature 35 ml of FBS-containing medium (complete medium) are added. The supernatant of cells is discarded and 300 μl of transfection complex-containing complete medium added. Cells are incubated in a CO2 incubator for three to four days before further processing.

### Fixation and Post-Processing of Cells

Ranges of concentrations and volumes are given where cell type-dependent optimization is required. Three to four days after transfection cells are fixed using 200 μl of 4% paraformaldehyde (PFA) for 10-20 minutes at room temperature. Cells are permeabilized using Triton X-100/PBS at a concentration range between 0.02 to 0.06% for 10 min at room temperature. In another embodiment cells are permeabilized with chilled acetone, digitonin, or with alternative permeabilization agents. After permeabilization cells are washed with PBS and nonspecific binding sites blocked with Superblock (Pierce) or alternative blocking agents.

### In Situ Denaturation of Proteins in Test Cells

In another embodiment PFA-fixed cells are treated with guanidinium thiocyanate (GTC) or urea for 10 minutes to denature cellular proteins in situ. Wells are thoroughly washed with PBS for five to ten times to remove traces of the denaturing agent prior to immunolabeling. After denaturation nonspecific binding sites are blocked as described above. In another embodiment test cells are plated and processed in 384 or 1536 well plates.

Testing Antibody Specificity on Target-Depleted Cells with Hybridoma Supernatants

Plates of cells treated with siRNA against the antibody target or scrambled control RNA and post-processed as described above are incubated with dilutions of hybridoma supernatants in a range of dilutions and incubated for 1 h at room temperature. After three washes with PBS wells are incubated with a fluorescent-containing secondary antibody for 1 h at room temperature. Plates are subsequently washed three to four times with PBS. After the final wash azide (0.03%)-containing PBS is added and plates are stored at 4° C. until further processing. Data analysis and specificity scoring of hybridoma supernatants conducted by high content screening is described below.

### Data Analysis and Specificity Scoring

To identify target-specific hybridoma supernatants labelled knockdown and control cells are scanned on a high-content screening system, for example Opera LX or IN Cell 6000 high content screening imagers, and analyzed using appropriate high content imaging and analysis software. Wells containing knockdown cells with a 50% decrease in fluorescence intensity compared to control cells are selected and subcloned.

In another embodiment test cells are plated and processed in 384 or 1536 well plates.

### Testing Specificity of Affinity Binders on Target-Depleted Cells

In another embodiment gene silenced and control cells, fixed and post-processed as described above are incubated with combinatorial antibody libraries, antibody fragments, or libraries of alternative affinity binders and processed essentially as described above.

Cells (human, mouse, hamster etc.) are transfected with methods according to the state of the art, e.g., chemical-based transfection with cyclodextrin, calcium phosphate, dendrimers liposomes, or nanoparticles (with or without chemical or viral functionalization), or cationic polymers such as DEAE-dextran or polyethylenimine, non-chemical methods like electroporation, sono-poration, optical transfection, impalefection, particle-based methods like the gene gun, or magnetofection.

### Testing Specificity of Affinity Binders by Inducible Expression of Antibody Targets

In another embodiment the antibody target and a control gene, respectively are expressed conditionally in cells that do not express the antibody target or cells that are depleted of the antibody target. This approach is chosen where (i) test cells do not express the antibody target, (ii) depletion of an antibody targets affects viability, (iii) affinity binders show low affinity for the target, and (iv) affinity binders are screened against members of a protein family or related proteins.

### High Content Screening Analysis of Hybridoma Supernatants

Cells are transfected with target-specific and scrambled control siRNA as described previously and cultured for three days. Cells are fixed, permeabilized and labelled using appropriate dilutions of hybridoma supernatants or antibody libraries and subsequently analyzed for the following cell features: Nuclear area measurement, cell count per field, and fluorescence intensities. Plates are imaged and quantitatively analyzed for the percentage reduction in fluorescence. Wells with fluorescence intensities of at least 50% less than controls are scored positive.

### Results and Discussion

The difficulty of assessing unequivocally the specificity of antibodies and antibody mimetics poses a major challenge for the development of reliable diagnostic and therapeutic antibodies. The development of the hybridoma technology by Georges Köhler and Cesar Milstein in 1975 [37] prompted confidence that this technological advancement resolved the specificity problem of antibodies as the isolation of monoclonal cultures of antibody-secreting plasma B cells provides a source for affinity-matured lymphocytes. However, seminal evidence of the multi specificity (polyspecificity, degeneracy [38,39]) of antibody binding greatly challenged the view of an inherent monospecificity of monoclonal antibodies. A monoclonal antibody (IgG1b12), isolated from a HIV-infected person reacted with double-stranded DNA (dsDNA), histones, and centromere B in addition to binding the viral gp120 protein [40]. The cross-reactivity of antibodies has been reported for various antigens [41-43]. About twenty percent of monoclonal antibodies, derived from 140 IgG-expressing memory B cells in normal human subjects were found to be reactive with a set of diverse ligands, including insulin, lipopolysaccharides and dsDNA [44]. Most remarkably, in 2010, Mouquet et al. reported that up to seventy-five percent of a total of 134 monoclonal anti-HIV-gp140 antibodies cloned from six patients were polyreactive [45]. These data unambiguously demonstrate the critical importance of antibody validation to warrant the target specificity of antibodies and alternative affinity binders.

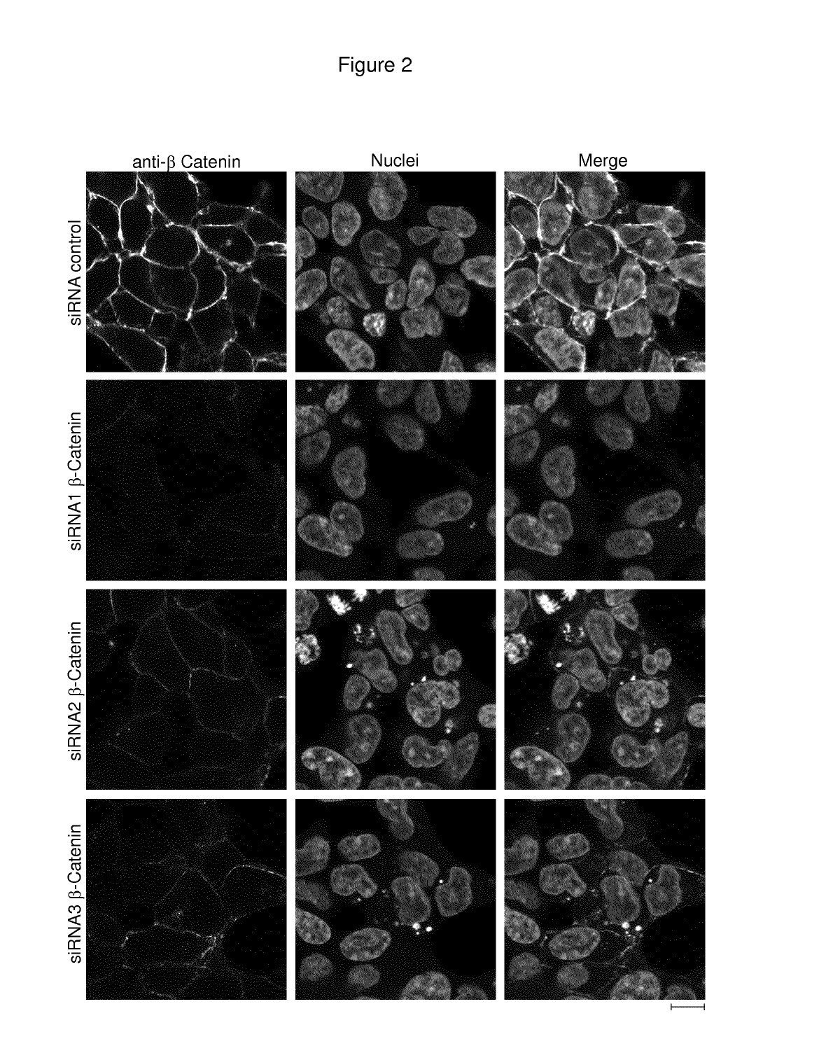

We here propose a strategy for the identification of superior antibodies by interrogating the specificity of antibodies using high-throughput screening. The use of reverse phase protein arrays (RPPA) in combination with RNAi has recently been suggested as a possibility to test antibody specificity [7]. We suggest that immunofluorescence analysis with whole mammalian cell lines provides a superior method for antibody specificity testing when compared to lysates [7] due to two main features of cell-based assays, the compartmentalization and structural organization of proteins. As depicted in FIG. 1, an antibody raised against Early Endosome Antigen 1 (EEA1) shows a discrete punctate pattern in control cells. EEA1 is expressed in early endosomes and serves as a marker of this tubular-vesicular network. Therefore the labelling of EEA1 by a specific immunoglobulin is associated with a discrete signal which is due to the compartmentalization of early endosomes in the cell. Upon deletion of EEA1 protein expression by siRNAs against EEA1 mRNA the anti-EEA1 antibody ceased to bind (FIG. 1). The complete lack of antibody binding in EEA1-silenced cells confirms the specificity of the anti-EEA1 antibody. The structural organization of proteins in whole cells is depicted in FIG. 1 by labelling of the tubulin network with an anti-Tubulin antibody. An antibody against tubulin is predicted to reveal the tubular structure of its protein target. Structural organization of proteins is therefore a second important feature of cell-based assays which provide additional criteria to validate antibody specificity. Other examples for compartmentalization and structural organization are depicted in FIGS. 2-4 and 6-10.

The efficacy of RNAi is critically dependent on the siRNA sequence. However, as the optimal siRNA can currently not be predicted and has to be tested empirically, five or more siRNAs are generally designed per target using established algorithms [22] and tested individually. As a first approximation of the efficacy of gene silencing the mRNA content is determined by quantitative real-time PCR (qPCR) after gene silencing. Scrambled RNA or siRNA against an unrelated target serves as a negative control. The level of knockdown for the siRNAs against β-catenin in FIG. 2 was 95%, 90% and 70% for siRNA1, 2 and 3, respectively. In case commercial antibodies are available the efficacy of RNAi can be preferably tested on the protein level. FIG. 2 depicts the degree of antibody labelling after silencing of β-catenin, a protein that is expressed at the plasma membrane. Quantification of the normalized residual fluorescence signal (NRF) with analysing software Volocity yielded NRF values of 1, 0.05, 0.15, 0.25 for siRNA control, siRNA1 b-catenin, siRNA2 b-catenin and siRNA3 b-catenin, respectively.

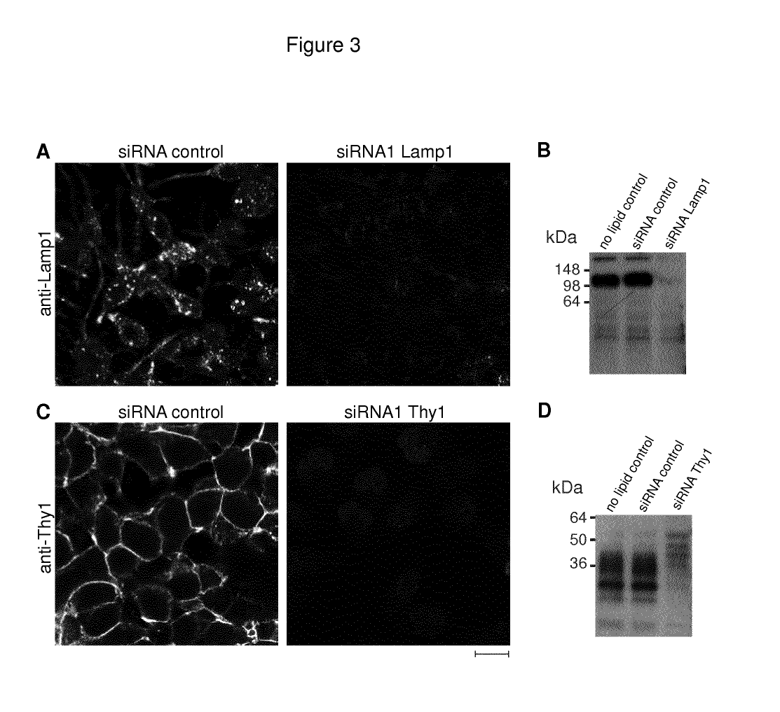

Importantly, the specificity of antibodies determined by immunocytochemistry can be independently verified by Western blotting. In FIG. 3B antibody labelling of a proteins blotted onto a PVDF membrane with an antibody against Lamp1 (Santa Cruz, clone 1D4B) shows a >95% loss of signal and thus confirms the specificity of the antibody. In analogy, the RNAi-mediated loss of fluorescence in whole cells can be verified by immunoblotting (FIG. 3C,D).

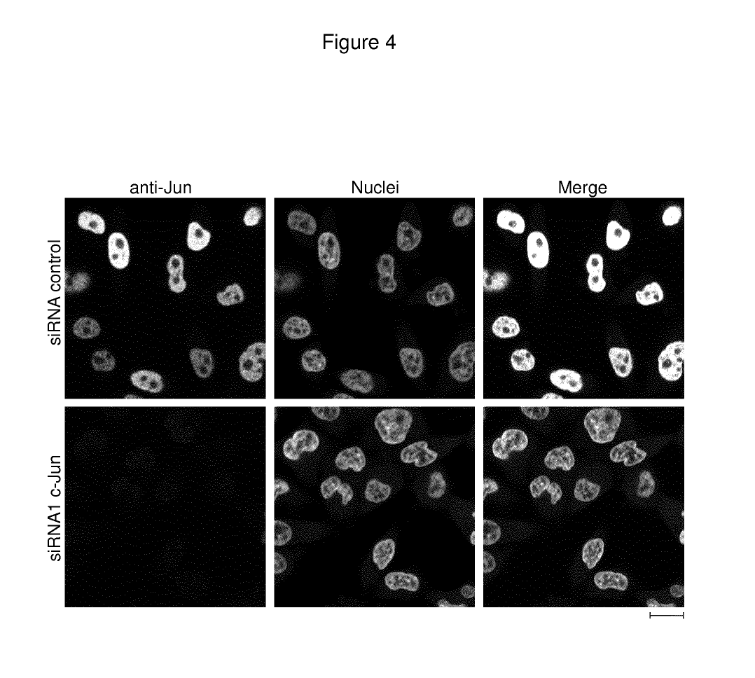

As shown in FIG. 4, a monoclonal antibody against c-Jun (BD Transduction labs, clone 3/Jun) labels nuclei and shows colocalization in the merged images. siRNA against c-Jun completely abrogates the nuclear label.

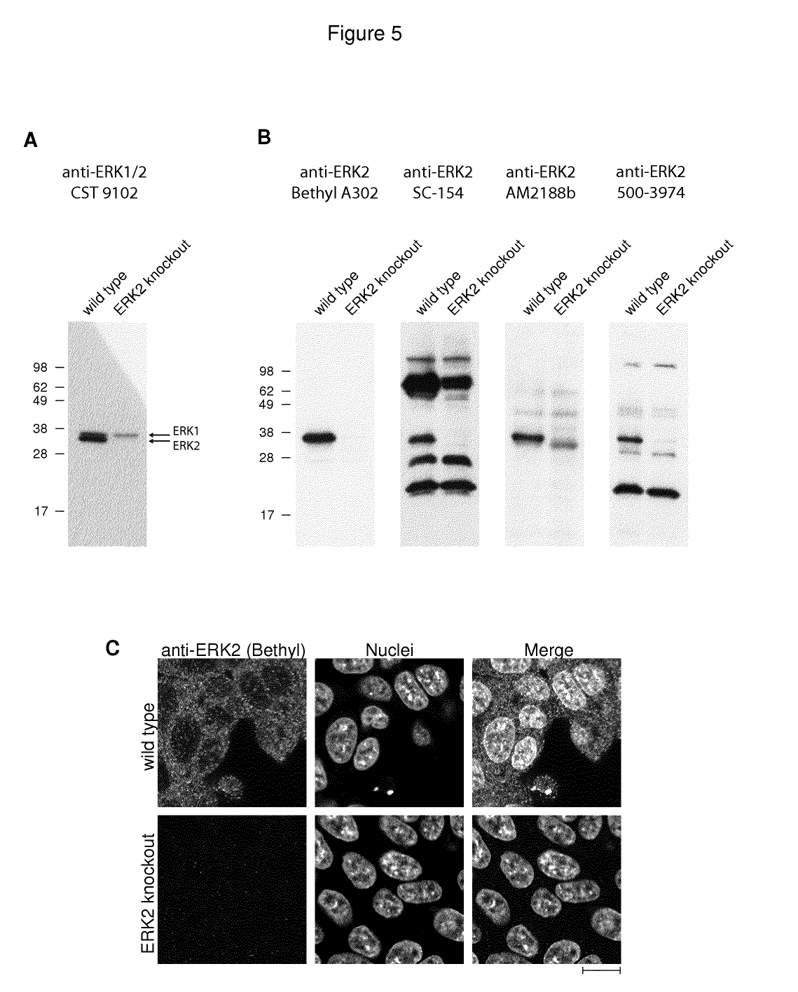

In addition to RNAi, the specificity of antibodies can be alternatively checked by deletion of the antibody target-encoding gene. CRISPR/Cas9-derived RNA-guided nucleases are DNA targeting systems, which are being harnessed for gene editing purposes in model organisms and cell lines. The versatility of genome editing by CRISPR/Cas9 and Talen has recently been reported in a number of publications [46-51]. The two components required to target a specific gene locus, a guide RNA (gRNA) and the CRISPR associated (Cas) nuclease Cas9 can be expressed in cells from the same or from separate vectors. The gRNA/Cas9 complex is recruited to the target sequence of the genomic DNA. Binding of the gRNA/Cas9 complex to genomic DNA triggers a cut of both strands of DNA causing a double strand break (DSB). A DSB can be repaired by the Non-Homologous End Joining (NHEJ) DNA repair pathway which often results in inserts/deletions (InDels) at the DSB site. This can lead to frameshifts and/or premature stop codons, effectively disrupting the open reading frame of the targeted gene. To delete the p42 MAPK (ERK2) gene in HAP1 cells a gRNA was designed which causes a frameshift in exon 1 of the EKR2 gene. The transfected cells were subcloned and complete knockout clones identified by PCR and Sanger sequencing. To test the specific deletion of ERK2, but not of ERK1, lysates from wild-type (wt) and ERK2-knockout cells were separated by electrophoresis, blotted onto PVDF membranes and labelled with antiserum against total ERK (p44/p42 MAPK (ERK1/2), CST, Cat #9102). As shown in FIG. 5A, the lower band (ERK2), but not the upper band (ERK1) was deleted in HAP1 cells, thus confirming that HAP1-ERK2 knockout (ko) cells are a suitable model to test the specificity of commercially available anti-ERK2 antibodies. Lysates containing equal amounts of protein from wt and ERK2 ko cells were subsequently separated by Western blotting and the PVDF membranes labelled with anti-ERK2 antibodies from Bethyl (Cat # A302-061A), Santa Cruz (Sc-154), Abgent (AM2188b) and Abbomax (500-3974). Strikingly, one out of four anti-ERK2 antibodies tested specific, whereas all other antibodies showed variable degrees of cross-reactivity with unidentified proteins (FIG. 5B). The specific antibody, anti-ERK2 (Bethyl) was subsequently used to label fixed and permeabilized HAP1 cells (FIG. 5C). In analogy to the WB results in FIG. 5B lysates from wt, but not from ko cells showed a cytoplasmic ERK2 label (FIG. 5B).

Linear peptides and recombinant proteins are widely used as immunogens to develop antibodies against specific protein targets. As the 3D structure of the vast majority of proteins is unknown, the characterization of antibodies in respect to their specificity and binding properties poses huge challenges. The binding epitope an antibody might be hidden (cryptic) in the native conformation of the protein in vivo, in which case high-content screening of antibodies in cell-based assays might fail to characterize the antibody as it does not binding to the target. We here present a method to interrogate the specificity of antibodies against cryptic and discontinuous epitopes in high-throughput mode, a screening strategy which allows the characterization of antibodies which may otherwise be missed during the course of high-throughput screening.

Whilst the specificity of an antibody against the mitochondrial protein Death-Associated Protein 3 (DAP3) can be validated in lysates of HeLa cells by Western blotting under denaturing conditions (FIG. 6A), the antibody fails to detect DAP3 in fixed cells by immunocytochemistry (data not shown). As the Western blot validation clearly confirms the presence of DAP3 in HeLa cells as well as the specificity of the anti-DAP3 antibody (Everest Biotech, EB05427) used, we concluded that the corresponding epitope might be cryptic under native conditions. To investigate this assumption we denatured fixed HeLa cells and subsequently labelled the cells with anti-DAP3 antibody. As shown in FIG. 6B protein denaturation revealed the typical mitochondrial label of DAP3 in immunocytochemistry. In situ denaturation of fixed cells therefore constitutes a powerful method to validate the specificity of antibodies against cryptic epitopes.

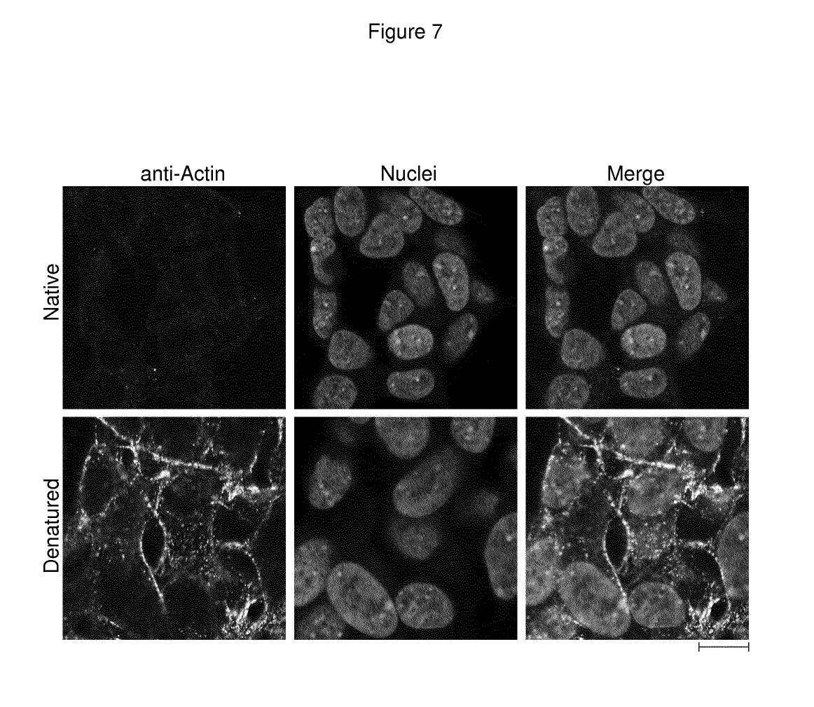

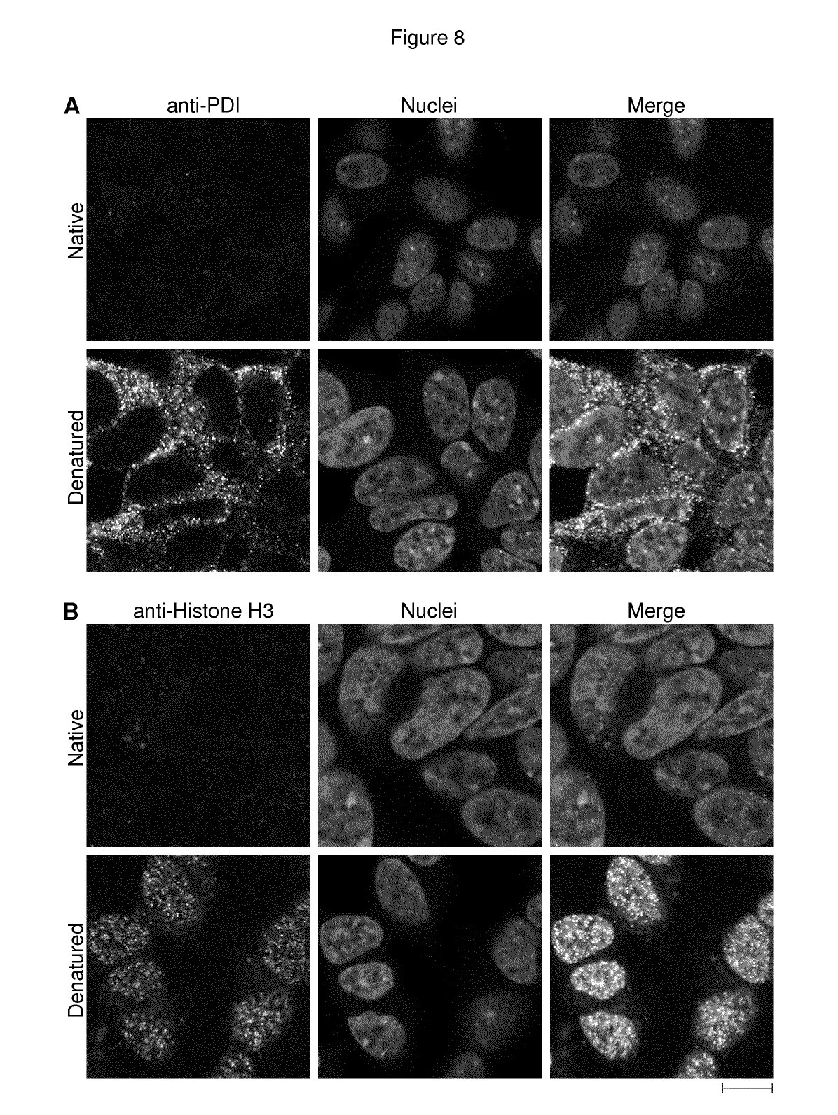

We then tested in a more systematic way whether other antibodies bind to cryptic epitopes and may therefore be missed during specificity testing of antibodies under standard conditions. In fact, in addition to the anti-DAP3 antibody (FIG. 6), a monoclonal anti-Actin antibody (Abcam, clone 2Q1055, FIG. 7), a monoclonal anti-Protein Disulfid Isomerase (PDI) antibody (CST, clone C81H6, FIG. 8A), and a monoclonal anti-Histone H3 antibody (CST, clone D1H2, FIG. 8B) bound to their corresponding protein target under denaturing, but not under native conditions.

In situ denaturation furthermore facilitates the identification of antibodies that bind to the native, but not the denatured protein. A monoclonal antibody against Nucleoporin 98 kDa (CST, clone C39A3) detects the double membrane surrounding the cell nucleus in SHSY5Y cells under native conditions (FIG. 9). However, under denaturing conditions the antibody ceased to bind which indicates that the epitope is sensitive to denaturation, a typical feature of conformational epitopes. Discontinuous conformational epitopes, which represent about ninety percent of all B cell epitopes are impossible to predict unless knowledge of the antigen's molecular structure is known [52].

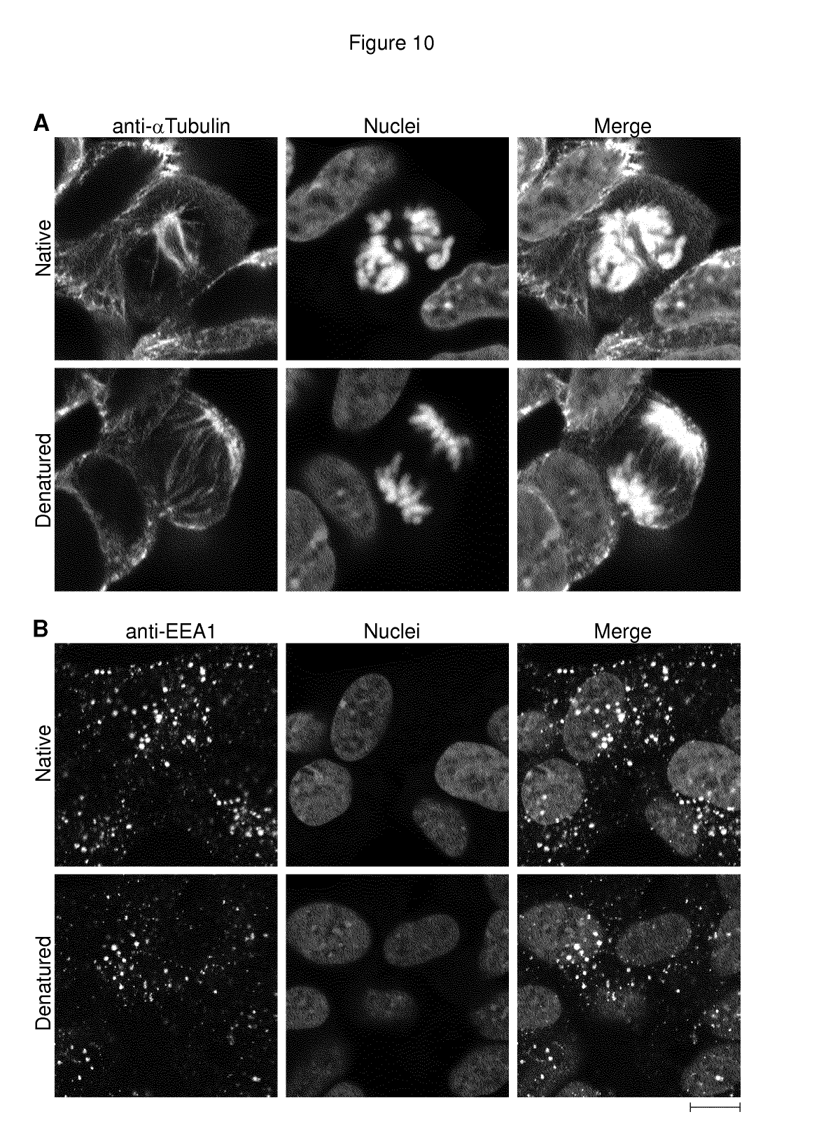

A third category of antibodies is unaffected by protein conformational changes (FIG. 10). A monoclonal antibody against α-Tubulin (GenTex, clone GT114) and a monoclonal antibody against EEA1 (CST, clone C45B10) bind to the corresponding targets in presence and absence of denaturing agents (FIG. 10).

The frequent identification of antibodies that do not recognize the protein target under native conditions (FIGS. 6-8) is surprising and to our knowledge unacknowledged. As ELISA assays with recombinant proteins and Western blotting are the most frequent assays used to characterize antibodies, the cryptic nature of the epitope may not be recognized with either method. This poses unappreciated problems for in vitro screening of antibodies libraries, since a substantial number of binders to cryptic epitopes remain undetected, unless the conformation of the epitope is modulated in presence and absence of denaturing conditions as shown in FIGS. 6-10. We therefore established a novel high-throughput screening module, the Epitope Conformation Switch™ (ECS™) assay to (a) identify and (b) validate antibodies that recognize cryptic and discontinuous conformational epitopes (FIG. 11). The ECS™ assay provides vital information about the nature of epitope binding of antibodies and leads to identification of antibodies that bind to (a) cryptic, (b) conformational and (c) invariant non-conformational epitopes (FIG. 11A). A triage for the identification and characterization of target-specific antibodies is shown in FIG. 11B.

A schematic representation of distinct stages for the production of monoclonal antibodies is shown in FIG. 12. After immunization of the host animal with recombinant protein or peptide (A), plasma B cells are isolated, fused with myeloma (B cell cancer) cells and transferred into wells of a 96-well plate (B). The fused cells, termed hybridoma cells, are typically grown for two weeks as a heterogeneous mix of cells, comprising antibody secreting and non-secreting cells, non-fused lymphocytes and myeloma cells. The identification and selection of antibody-secreting (producer) cells (C) is limited to a period of one to maximal two weeks, when the plated cells reach confluence, stop dividing and start deteriorating. During this phase, hybridoma supernatants are retrieved and analyzed in regards to antibody isotypes and affinities. Subsequently, selected antibody-producing hybridoma cells are subcloned to obtain monoclonal hybridoma cells (D) and monoclonal antibodies are isolated and purified (E). The identification of target-specific antibodies by use the proposed screening triage (FIG. 11B) at stage C of the antibody production scheme (FIG. 12) allows enough time for the complete characterization of binders as all assays are amenable to high-throughput screening.

In summary, when used in combination with the genetic depletion of antibody targets, the ECS assay is a powerful high-throughput screening module that facilitates the identification and validation of antibodies against cryptic, discontinuous and invariant epitopes.

### REFERENCES

1. Grimsey N L, Goodfellow C E, Scotter E L, Dowie M J, Glass M, Graham E S: Specific detection of CB(1) receptors; cannabinoid CB(1) receptor antibodies are not all created equal! J Neurosci Methods 2008, 171:78-86.
2. Couchman J R: Commercial Antibodies: The Good, Bad, and Really Ugly. Journal of Histochemistry & Cytochemistry 2009, 57:7-8.
3. Jensen B C, Swigart P M, Simpson P C: Ten commercial antibodies for alpha-1-adrenergic receptor subtypes are nonspecific. Naunyn Schmiedebergs Arch Pharmacol 2009, 379:409-412.
4. Jositsch G, Papadakis T, Haberberger R V, Wolff M, Wess J, Kummer W: Suitability of muscarinic acetylcholine receptor antibodies for immunohistochemistry evaluated on tissue sections of receptor gene-deficient mice. Naunyn Schmiedebergs Arch Pharmacol 2009, 379:389-395.
5. Pradidarcheep W, Stallen J, Labruyere W T, Dabhoiwala N F, Michel M C, Lamers W H: Lack of specificity of commercially available antisera against muscarinergic and adrenergic receptors. Naunyn Schmiedebergs Arch Pharmacol 2009, 379:397-402.
6. Bordeaux J, Welsh A W, Agarwal S, Killiam E, Baquero M T, Hanna J A, Anagnostou V K, Rimm D L: Antibody validation. Biotechniques 2010, 48:197-209.
7. Mannsperger H A, Uhlmann S, Schmidt C, Wiemann S, Sahin O, Korf U: RNAi-based validation of antibodies for reverse phase protein arrays. Proteome Sci 2010, 8.
8. Stadler C, Hjelmare M, Neumann B, Jonasson K, Pepperkok R, Uhlen M, Lundberg E: Systematic validation of antibody binding and protein subcellular localization using siRNA and confocal microscopy. J Proteomics 2012, 75:2236-2251.
9. Shalem O, Sanjana N E, Hartenian E, Shi X, Scott D A, Mikkelsen T S, Heckl D, Ebert B L, Root D E, Doench J G, Zhang F: Genome-Scale CRISPR-Cas9 Knockout Screening in Human Cells. Science 2014, 343:84-87.
10. Qi L S, Larson M H, Gilbert L A, Doudna J A, Weissman J S, Arkin A P, Lim W A: Repurposing CRISPR as an RNA-Guided Platform for Sequence-Specific Control of Gene Expression. Cell 2013, 152:1173-1183.
11. Carmell M A, Xuan Z Y, Zhang M Q, Hannon G J: The Argonaute family: tentacles that reach into RNAi, developmental control, stem cell maintenance, and tumorigenesis. Genes Dev 2002, 16:2733-2742.
12. Karube Y, Tanaka H, Osada H, Tomida S, Tatematsu Y, Yanagisawa K, Yatabe Y, Takamizawa J, Miyoshi S, Mitsudomi T, Takahashi T: Reduced expression of Dicer associated with poor prognosis in lung cancer patients. Cancer Sci 2005, 96:111-115.
13. Merritt W M, Lin Y G, Han L Y, Kamat A A, Spannuth W A, Schmandt R, Urbauer D, Pennacchio L A, Cheng J, Nick A M, Deavers M T, Mourad-Zeidan A, Wang H, Mueller P, Lenburg M E, Gray J W, Mok S, Birrer M J, Lopez-Berestein G, Coleman R L, Bar-Eli M, Sood A K: Dicer, Drosha, and Outcomes in Patients with Ovarian Cancer. N Engl J Med 2008, 359:2641-2650.
14. Melo S A, Ropero S, Moutinho C, Aaltonen L A, Yamamoto H, Calin G A, Rossi S, Fernandez A F, Carneiro F, Oliveira C, Ferreira B, Liu C G, Villanueva A, Capella G, Schwartz S, Shiekhattar R, Esteller M: A TARBP2 mutation in human cancer impairs microRNA processing and DICER1 function. Nat Genet 2009, 41:365-370.
15. Keitel T, Kramer A, Wessner H, Scholz C, SchneiderMergener J, Hohne W: Crystallographic analysis of anti-p24 (HIV-1) monoclonal antibody cross-reactivity and polyspecificity. Cell 1997, 91:811-820.
16. Kramer A, Keitel T, Winkler K, Stocklein W, Hohne W, SchneiderMergener J: Molecular basis for the binding promiscuity of an anti-p24 (HIV-1) monoclonal antibody. Cell 1997, 91:799-809.
17. Van Regenmortel M H V: From absolute to exquisite specificity. Reflections on the fuzzy nature of species, specificity and antigenic sites. J Immunol Methods 1998, 216:37-48.
18. Pinilla C, Martin R, Gran B, Appel J R, Boggiano C, Wilson D B, Houghten R A: Exploring immunological specificity using synthetic peptide combinatorial libraries. Curr Opin Immunol 1999, 11:193-202.
19. Gras-Masse H, Georges B, Estaquier J, Tranchand-Bunel D, Tartar A, Druilhe P, Auriault C: Convergent peptide libraries, or mixotopes, to elicit or to identify specific immune responses. Curr Opin Immunol 1999, 11:223-228.
20. Fire A, Xu S Q, Montgomery M K, Kostas S A, Driver S E, Mello C C: Potent and specific genetic interference by double-stranded RNA in Caenorhabditis elegans. Nature 1998, 391:806-811.
21. Elbashir S M, Harborth J, Lendeckel W, Yalcin A, Weber K, Tuschl T: Duplexes of 21-nucleotide RNAs mediate RNA interference in cultured mammalian cells. Nature 2001, 411:494-498.
22. Reynolds A, Leake D, Boese Q, Scaringe S, Marshall W S, Khvorova A: Rational siRNA design for RNA interference. Nature Biotechnology 2004, 22:326-330.
23. Mortimer G M, Butcher N J, Musumeci A W, Deng Z J, Martin D J, Minchin R F: Cryptic Epitopes of Albumin Determine Mononuclear Phagocyte System Clearance of Nanomaterials. Acs Nano 2014, 8:3357-3366.
24. Poli F, Benazzi E, Innocente A, Nocco A, Cagni N, Gianatti A, Fiocchi R, Scalamogna M: Heart transplantation with donor-specific antibodies directed toward denatured HLA-A*02:01: a case report. Hum Immunol 2011, 72:1045-1048.
25. Vojtesek B, Dolezalova H, Lauerova L, Svitakova M, Havlis P, Kovarik J, Midgley C A, Lane D P: Conformational-Changes in P53 Analyzed Using New Antibodies to the Core Dna-Binding Domain of the Protein. Oncogene 1995, 10:389-393.
26. Garrett T P J, Burgess A W, Gan H K, Luwor R B, Cartwright G, Walker F, Orchard S G, Clayton A H A, Nice E C, Rothacker J, Catimel B, Cavenee W K, Old L J, Stockert E, Ritter G, Adams T E, Hoyne P A, Wittrup D, Chao G, Cochran J R, Luo C, Lou M Z, Huyton T, Xu Y B, Fairlie W D, Yao S G, Scott A M, Johns T G: Antibodies specifically targeting a locally misfolded region of tumor associated EGFR. Proc Natl Acad Sci USA 2009, 106:5082-5087.
27. Scardino A, Gross D A, Alves P, Schultze J L, Graff-Dubois S, Faure O, Tourdot S, Chouaib S, Nadler L M, Lemonnier F A, Vonderheide R H, Cardoso A A, Kosmatopoulos K: HER-2/neu and hTERT cryptic epitopes as novel targets for broad spectrum tumor immunotherapy. J Immunol 2002, 168:5900-5906.
28. Oaks M, Michel K, Sulemanjee N Z, Thohan V, Downey F X: Practical value of identifying antibodies to cryptic HLA epitopes in cardiac transplantation. J Heart Lung Transplant 2014, 33:713-720.
29. Wang D N, Bhat R, Sobel R A, Huang W, Wang L X, Olsson T, Steinman L: Uncovering Cryptic Glycan Markers in Multiple Sclerosis (MS) and Experimental Autoimmune Encephalomyelitis (EAE). Drug Dev Res 2014, 75:172-188.
30. Moudgil K D, Sercarz E E: Understanding crypticity is the key to revealing the pathogenesis of autoimmunity. Trends Immunol 2005, 26:355-359.
31. Lanzavecchia A: How Can Cryptic Epitopes Trigger Autoimmunity. J Exp Med 1995, 181:1945-1948.
32. Lehmann P V, Forsthuber T, Miller A, Sercarz E E: Spreading of T-Cell Autoimmunity to Cryptic Determinants of An Autoantigen. Nature 1992, 358:155-157.
33. Sercarz E E: Driver clones and determinant spreading. Journal of Autoimmunity 2000, 14:275-277.
34. Xu J S, Rodriguez D, Kim J J, Brooks P C: Generation of monoclonal antibodies to cryptic collagen sites by using subtractive immunization. Hybridoma 2000, 19:375-385.
35. Aguilar H N, Zielnik B, Tracey C N, Mitchell B F: Quantification of Rapid Myosin Regulatory Light Chain Phosphorylation Using High-Throughput In-Cell Western Assays: Comparison to Western Immunoblots. Plos One 2010, 5.
36. Hotzel I, Theil F P, Bernstein L J, Prabhu S, Deng R, Quintana L, Lutman J, Sibia R, Chan P, Bumbaca D, Fielder P, Carter P J, Kelley R F: A strategy for risk mitigation of antibodies with fast clearance. Mabs 2012, 4:753-760.
37. Kohler G, Milstein C: Continuous Cultures of Fused Cells Secreting Antibody of Predefined Specificity. Nature 1975, 256:495-497.
38. James L C, Tawfik D S: Structure and kinetics of a transient antibody binding intermediate reveal a kinetic discrimination mechanism in antigen recognition. Proc Natl Acad Sci USA 2005, 102:12730-12735.
39. Eisen H N, Chakraborty A K: Evolving concepts of specificity in immune reactions. Proc Natl Acad Sci USA 2010, 107:22373-22380.
40. Haynes B F, Fleming J, St Clair E W, Katinger H, Stiegler G, Kunert R, Robinson J, Scearce R M, Plonk K, Staats H F, Ortel T L, Liao H X, Alam S M: Cardiolipin polyspecific autoreactivity in two broadly neutralizing HIV-1 antibodies. Science 2005, 308:1906-1908.
41. Schubert D, Jobe A, Cohn M: Mouse Myelomas Producing Precipitating Antibody to Nucleic Acid Bases And/Or Nitrophenyl Derivatives. Nature 1968, 220:882-&.
42. Michaelidis M C, Eisen H N: Strange Cross-Reaction of Menadione (Vitamin-K3) and 2,4-Dinitrophenyl Ligands with A Myeloma Protein and Some Conventional Antibodies. J Exp Med 1974, 140:687-702.
43. Rosenstein. R W, Richards F F, Armstron. MY, Konigsbe. WH, Musson R A: Contact Regions for Dinitrophenyl and Menadione Haptens in An Immunoglobulin Binding More Than One Antigen. Proc Natl Acad Sci USA 1972, 69:877-&.
44. Tiller T, Tsuiji M, Yurasov S, Velinzon K, Nussenzweig M C, Wardemann H: Autoreactivity in human IgG(+) memory B cells. Immunity 2007, 26:205-213.
45. Mouquet H, Scheid J F, Zoller M J, Krogsgaard M, Ott R G, Shukair S, Artyomov M N, Pietzsch J, Connors M, Pereyra F, Walker B D, Ho D D, Wilson P C, Seaman M S, Eisen H N, Chakraborty A K, Hope T J, Ravetch J V, Wardemann H, Nussenzweig M C: Polyreactivity increases the apparent affinity of anti-HIV antibodies by heteroligation. Nature 2010, 467:591-U117.
46. Sanjana N E, Cong L, Zhou Y, Cunniff M M, Feng G P, Zhang F: A transcription activator-like effector toolbox for genome engineering. Nat Protoc 2012, 7:171-192.
47. Fu Y F, Foden J A, Khayter C, Maeder M L, Reyon D, Joung J K, Sander J D: High-frequency off-target mutagenesis induced by CRISPR-Cas nucleases in human cells. Nat Biotechnol 2013, 31: 822-+.
48. Yang H, Wang H Y, Shivalila C S, Cheng A W, Shi L Y, Jaenisch R: One-Step Generation of Mice Carrying Reporter and Conditional Alleles by CRISPR/Cas-Mediated Genome Engineering. Cell 2013, 154:1370-1379.
49. Ran F A, Hsu P D, Lin C Y, Gootenberg J S, Konermann S, Trevino A E, Scott D A, Inoue A, Matoba S, Zhang Y, Zhang F: Double Nicking by RNA-Guided CRISPR Cas9 for Enhanced Genome Editing Specificity. Cell 2013, 154:1380-1389.
50. Horvath P, Barrangou R: CRISPR/Cas, the Immune System of Bacteria and Archaea. Science 2010, 327:167-170.
51. Mali P, Esvelt K M, Church G M: Cas9 as a versatile tool for engineering biology. Nat Methods 2013, 10:957-963.
52. Tomar N, De R K: Immunoinformatics: an integrated scenario. Immunology 2010, 131:153-168.

1-16. (canceled)

17. A method for screening a plurality of cells for their capacity to secrete an antibody or an alternative affinity binder with affinity and/or specificity for a target antigen, which method comprises comparing the affinity and/or specificity of each antibody or alternative affinity binder produced from the plurality of cells using a protocol comprising of the following steps:
(i) analyzing binding of the antibody, or the alternative affinity binder, to a target antigen expressed by an antigen-expressing cell;
(ii) analyzing binding of the antibody, or the alternative affinity binder, to a target antigen expressed by an equivalent antigen-expressing cell, in which the target antigen-encoding gene has been transcriptionally silenced, and/or deleted; and
(iii) comparing the binding reactions of the antibody, or the alternative affinity binder, in steps (i) and (ii).

18. The method of claim 17, wherein transcriptional silencing or deletion of the target antigen is accomplished by RNA-mediated interference (RNAi) (transcriptional silencing of the target antigen), CRISPR Cas technology (deletion of the target antigen-encoding gene), Talen technology (deletion of the target antigen-encoding gene), and/or Zn finger nuclease technology (deletion of the target antigen-encoding gene).

19. The method of claim 17, wherein binding of the antibody or the alternative affinity binder in steps (i) and/or (ii) is analyzed directly on or in the antigen-expressing cell.

20. The method of claim 17, wherein the binding in steps (i) and/or (ii) is studied in a lysate prepared from the antigen-expressing cell.

21. The method of claim 17, wherein the antigen-expressing cell is generated by introducing a gene encoding the target antigen under the control of an inducible promoter.

22. The method of claim 17, further comprising treating at least one antigen-expressing cell, or the antigens expressed by said cell, with a denaturing agent before the binding of the antibody or the alternative affinity binder in steps (i) and/or (ii) is analyzed.

23. The method of claim 22, further comprising identifying antibodies or affinity binders that are a) specific and bind to cryptic epitopes of a target inaccessible in its native conformation, b) specific and bind conformational epitopes, and/or c) specific and are invariant to conformational changes of the epitope.

24. The method of claim 17, further comprising providing one or more antibodies, or one or more alternative affinity binders, by a library of cells.

25. The method of claim 24, wherein the library of cells is a library of hybridoma cells, a naive antibody library, a synthetic (combinatorial) antibody library, and/or a synthetic (combinatorial) library of alternative affinity binders.

26. The method of claim 17, wherein the cell line is a hybridoma.

27. The method of claim 26, further comprising producing a plurality of hybridoma cells by immunizing an animal with the target antigen, isolating a plurality of antibody-producing cells from the immunised animal, and fusing the plurality of antibody-producing cells from the immunised animal and fusing the plurality of antibody-producing cells with an immortal cell type.

28. The method of claim 17, wherein the binding of the antibody or alternative affinity binder is analysed by immunofluorescence, western blotting, enzyme-linked immunosorbent assay (ELISA), and/or surface plasmon resonance (SPR) based technology.

29. The method of claim 17, further comprising using the antibody or alternative affinity binder for a therapeutic purpose, scientific purpose, diagnostic purpose, and/or forensic purpose.

30. A method for selecting a cell line which produces an antibody or an alternative affinity binder that has affinity and/or specificity for a target antigen which method comprises screening a plurality of cells by a protocol comprising of the following steps:
(i) analyzing binding of the antibody, or the alternative affinity binder, to a target antigen expressed by an antigen-expressing cell;
(ii) analyzing binding of the antibody, or the alternative affinity binder, to a target antigen expressed by an equivalent antigen-expressing cell, in which the target antigen has been transcriptionally silenced, and/or deleted;
(iii) comparing the binding reactions of the antibody, or the alternative affinity binder, in steps (i) and (ii); and
(iv) selecting an antibody or an alternative affinity binder which shows a reduced or absent binding in step (ii) compared to step (i).

31. The method of claim 30, wherein transcriptional silencing or deletion of the target antigen is accomplished by RNA-mediated interference (RNAi) (transcriptional silencing of the target antigen), CRISPR Cas technology (deletion of the target antigen-encoding gene), Talen technology (deletion of the target antigen-encoding gene), and/or Zn finger nuclease technology (deletion of the target antigen-encoding gene).

32. The method of claim 30, wherein binding of the antibody or the alternative affinity binder in steps (i) and/or (ii) is analyzed directly on or in the antigen-expressing cell.

33. The method of claim 30, wherein the binding in steps (i) and/or (ii) is studied in a lysate prepared from the antigen-expressing cell.

34. The method of claim 30, wherein the antigen-expressing cell is generated by introducing a gene encoding the target antigen under the control of an inducible promoter.

35. The method of claim 30, further comprising treating at least one antigen-expressing cell, or the antigens expressed by said cell, with a denaturing agent before the binding of the antibody or the alternative affinity binder in steps (i) and/or (ii) is analyzed.

36. The method of claim 35, further comprising identifying antibodies or affinity binders that are a) specific and bind to cryptic epitopes of a target inaccessible in its native conformation, b) specific and bind conformational epitopes, and/or c) specific and are invariant to conformational changes of the epitope.

37. The method of claim 30, further comprising providing one or more antibodies, or one or more alternative affinity binders, by a library of cells.

38. The method of claim 37, wherein the library of cells is a library of hybridoma cells, a naive antibody library, a synthetic (combinatorial) antibody library, and/or a synthetic (combinatorial) library of alternative affinity binders.

39. The method of claim 30, wherein the cell line is a hybridoma.

40. The method of claim 39, further comprising producing a plurality of hybridoma cells by immunizing an animal with the target antigen, isolating a plurality of antibody-producing cells from the immunised animal, and fusing the plurality of antibody-producing cells from the immunised animal and fusing the plurality of antibody-producing cells with an immortal cell type.

41. The method of claim 30, wherein the binding of the antibody or alternative affinity binder is analysed by immunofluorescence, western blotting, enzyme-linked immunosorbent assay (ELISA), and/or surface plasmon resonance (SPR) based technology.

42. The method of claim 30, further comprising using the antibody or alternative affinity binder for a therapeutic purpose, scientific purpose, diagnostic purpose, and/or forensic purpose.

43. An antibody or alternative affinity binder produced by the method of any one of claims 17-42.
